(19) United States
(12) Patent Application Publication
(10) Pub. No.: US 2004/0267195 A1
(43) Pub. Date: Dec. 30, 2004

(54) Catheter balloon having visible marker

(76) Inventor: Arnoldo Currlin, Del Mar, CA (US)

Correspondence Address: FULWIDER PATTON LEE & UTECHT, LLP, HOWARD HUGHES CENTER, 6060 CENTER DRIVE, TENTH FLOOR, LOS ANGELES, CA, 90045, US

(21) Appl. No.: 10/602,850

(22) Filed: Jun. 24, 2003

(51) Int. Cl. A61M 31/00

(52) U.S. Cl. 604/103.1; 427/2.1; 623/1.11

(57) ABSTRACT

A balloon catheter, and a method of making the balloon catheter, having at least one visible mark which is visible from an outer surface of the balloon. The balloon catheter is configured for use as a dilatation catheter or stent delivery system, and the visible mark facilitates assembly of the balloon catheter or stent delivery system. In a presently preferred embodiment, the visible mark is an ink coating mark, a titanium oxide mark, or a scorch mark.

### BACKGROUND OF THE INVENTION

This invention generally relates to medical devices, and particularly to intravascular devices for therapeutic or diagnostic uses, such as balloon catheters.

In percutaneous transluminal coronary angioplasty (PTCA) procedures, a guiding catheter is advanced until the distal tip of the guiding catheter is seated in the ostium of a desired coronary artery. A guidewire is first advanced out of the distal end of the guiding catheter into the patient's coronary artery until the distal end of the guidewire crosses a lesion to be dilated. Then the dilatation catheter having an inflatable balloon on the distal portion thereof is advanced into the patient's coronary anatomy until the balloon of the dilatation catheter is properly positioned across the lesion. Once properly positioned, the dilatation balloon is inflated with fluid one or more times to a predetermined size at relatively high pressures (e.g. greater than 8 atmospheres) so that the stenosis is compressed against the arterial wall and the wall expanded to clear the passageway. Generally, the inflated diameter of the balloon is approximately the same diameter as the native diameter of the body lumen being dilated so as to complete the dilatation but not overexpand the artery wall. Substantial, uncontrolled expansion of the balloon against the vessel wall can cause trauma to the vessel wall. Additionally, characteristics of the balloon such as strength, compliance (rate of expansion), and profile are carefully tailored depending on the desired use of the balloon catheter, and the balloon material and manufacturing procedure are chosen to provide the desired balloon characteristics. After the balloon is finally deflated, blood flow resumes through the dilated artery and the dilatation catheter can be removed therefrom.

In such angioplasty procedures, there may be restenosis of the artery, i.e. reformation of the arterial blockage, which necessitates either another angioplasty procedure, or some other method of repairing or strengthening the dilated area. To reduce the restenosis rate and to strengthen the dilated area, physicians frequently implant a stent inside the artery at the site of the lesion. Stents may also be used to repair vessels having an intimal flap or dissection or to generally strengthen a weakened section of a vessel. Stents are usually delivered to a desired location within a coronary artery in a contracted condition on a balloon of a catheter which is similar in many respects to a balloon angioplasty catheter, and expanded to a larger diameter by expansion of the balloon. The balloon is deflated to remove the catheter and the stent left in place within the artery at the site of the dilated lesion. Stent covers on an inner or an outer surface of the stent have been used in, for example, the treatment of pseudo-aneurysms and perforated arteries, and to prevent prolapse of plaque.

To facilitate placement of the catheter at the desired location in the patient's vasculature, X-ray opaque (i.e., radiopaque) material is generally provided on conventional angioplasty catheters so that the physician can view the catheter under fluoroscopy. The radiopaque material is typically a metal marker band on the catheter shaft. For example, marker band(s) are typically provided on the inner tubular member of the shaft, to indicate the balloon. As an alternative to radiopaque marker bands on the catheter shaft, blending or depositing radiopaque material into or on the polymeric material of the catheter balloon has been suggested. Additionally, catheters visible to magnetic resonance imaging (MRI), also known as nuclear magnetic resonance imaging (NMR) systems, have been suggested for use during MRI scans of a patient. MRI scans are used to provide two-dimensional sectional images of a patient's internal body structures without exposing the patient to harmful radiation. During attachment of the balloon to the catheter shaft, the marker material on the inner tubular member of the shaft must be correctly aligned with a desired part of the balloon. However, one difficulty has been accurately aligning the balloon working length with the marker(s). What has been needed is a catheter balloon with improved manufacturability, providing improved catheter performance.

### SUMMARY OF THE INVENTION

This invention is directed to a balloon catheter, and a method of making the balloon catheter, having at least one visible mark which is visible from an outer surface of the balloon. The balloon catheter is configured for use as a dilatation catheter or stent delivery system, and the visible mark preferably facilitates assembly of the balloon catheter or stent delivery system.

A balloon catheter of the invention generally comprises an elongated shaft having a proximal shaft section, a distal shaft section, and at least one lumen, with a balloon on the distal shaft section. The shaft typically comprises an inner tubular member defining a guidewire lumen and an outer tubular member defining an inflation lumen, although a variety of shaft designs may be used. The balloon has a proximal skirt section and a distal skirt section secured to the shaft, so that an inflatable interior of the balloon is in fluid communication with the inflation lumen. The balloon inflates to an inflated configuration having an inflated working length, an inflated proximal tapered section between the inflated working length and the proximal skirt section, and an inflated distal tapered section between the inflated working length and the distal skirt section.

In one embodiment, the balloon has a proximal visible mark formed at the proximal end of the working length of the balloon, and a distal visible mark formed at the distal end of the working length of the balloon. The proximal and distal visible marks thus indicate the location of the balloon shoulders. In one aspect of the invention, during assembly of the balloon catheter, the proximal and distal visible marks at the balloon shoulders facilitate accurately aligning the shoulders of the balloon with markers (e.g., radiopaque markers) located on the portion of the elongated shaft extending through the balloon interior. In another aspect of the invention, after the balloon is secured to the shaft, the proximal and distal visible marks at the balloon shoulders facilitate accurately mounting a stent at a desired location on the balloon working length. Although discussed primarily in terms of the embodiment in which the balloon visible marks are at either end of the balloon working length, it should be understood that the balloon visible mark may be provided at a variety of suitable locations on the outer surface of the balloon, depending, for example, on the location of a marker on the portion of the shaft extending through the balloon interior.

In another embodiment, the visible mark is made on the edge of a wing of the balloon in a deflated configuration. The balloon in the deflated configuration has one or more wings wrapped around the balloon prior to being inflated in a patient's body lumen, thus providing a low profile configuration for introduction and advancement of the balloon catheter within the patient's body lumen. The visible mark on the edge of the wing(s) facilitates inspection of the balloon to determine for example the amount to which the balloon was twisted during subsequent processing of the balloon. For example, the balloon may twist as the balloon is inserted into a sheath during processing of the balloon or to prepare the balloon catheter for storage or shipment. Excessive twisting affects balloon performance, so the inspection thus ensures that disadvantageous twisting of the balloon is not present and any twisting is within specifications. Additionally, the visible mark on the edge of the wing(s) facilitates inspection of the balloon to determine the amount of pinching of the folded wings of the balloon by a stent mounted on the balloon. Excessive pinching can damage the balloon, and the inspection thus ensures that disadvantageous pinching of the balloon is not present.

The visible mark typically extends at least partially around the circumference of the balloon, and in one presently preferred embodiment extends completely around the circumference of the balloon to facilitate the technician's ability to view the mark. The visible mark is formed on the balloon either with the balloon in the inflated configuration or in the deflated configuration. In the embodiment having folded wings wrapped around the balloon in the deflated configuration, part of the outer surface of the balloon is covered by the folded wings. Therefore, a visible mark formed on the deflated balloon and extending completely around the circumference of the deflated balloon, extends only partially around the circumference of the inflated balloon.

In a presently preferred embodiment, the visible mark is selected from the group consisting of an ink coating mark, a titanium oxide mark, and a scorch mark. The visible mark is typically not radiopaque. The terminology “visible” mark as used herein should be understood to refer to a mark visible to the naked eye or by magnification. The scorch mark is preferably formed by exposing the outer surface of the balloon to laser radiation to scorch the balloon material. The laser scorching discolors the balloon material with minimal damage or affect to the balloon material or characteristics, and preferably without removing (e.g., vaporizing) the balloon material and thus without forming a channel or groove in the outer surface of the balloon. The laser scorching forms a permanent mark without requiring the addition of a coating, layer, or other additional material to the balloon, and therefore preferably has no affect on the compliance of the balloon. In an alternative embodiment, the visible mark is made by applying an ink coating onto the balloon, thereby providing highly visible marks without damaging the balloon material. In an alternative embodiment, titanium oxide (TiO) forms the visible mark. For example, the balloon polymeric material forming at least a layer of the balloon is loaded with (i.e., blended or otherwise combined with) titanium dioxide (TiO₂), and the titanium dioxide is then selectively reduced to form titanium oxide which changes the color of the material in the desired pattern. Specifically, exposing a portion of the titanium dioxide loaded balloon to a radiation source such as an ultraviolet laser converts the exposed titanium dioxide to titanium oxide which is visible to the naked eye as a light gray or black mark.

The balloon catheter of the invention having visible marks on the outer surface of the balloon facilitates assembly of the balloon catheter or stent delivery system. The visible marks provide improved manufacturability of the catheter and provide improved catheter performance. These and other advantages of the invention will become more apparent from the following detailed description of the invention and the accompanying exemplary drawings.

### BRIEF DESCRIPTION OF THE DRAWINGS

FIG. 1 is an elevational view, partially in section, of a stent delivery balloon catheter embodying features of the invention, with a proximal and a distal visible mark at either end of the working length of the balloon.

FIG. 2 is a transverse cross sectional view of the balloon catheter shown in FIG. 1, taken along line 2-2.

FIG. 3 is a transverse cross sectional view of the balloon catheter shown in FIG. 1, taken along line 3-3.

FIG. 4 illustrates a longitudinal cross sectional view of the balloon catheter of FIG. 1, with the balloon in an inflated configuration to expand the stent within the patient's body lumen.

FIG. 5 illustrates a distal end of a balloon catheter embodying features of the invention, with the balloon in a deflated configuration having wings wrapped therearound and a visible mark on the edge of the wings.

### DETAILED DESCRIPTION OF THE PREFERRED EMBODIMENTS

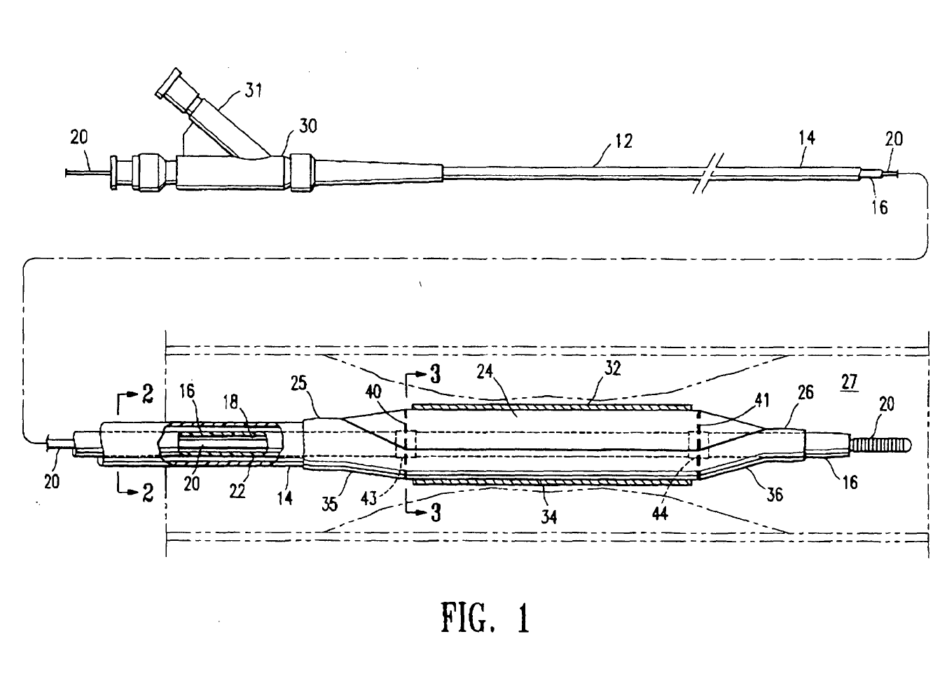

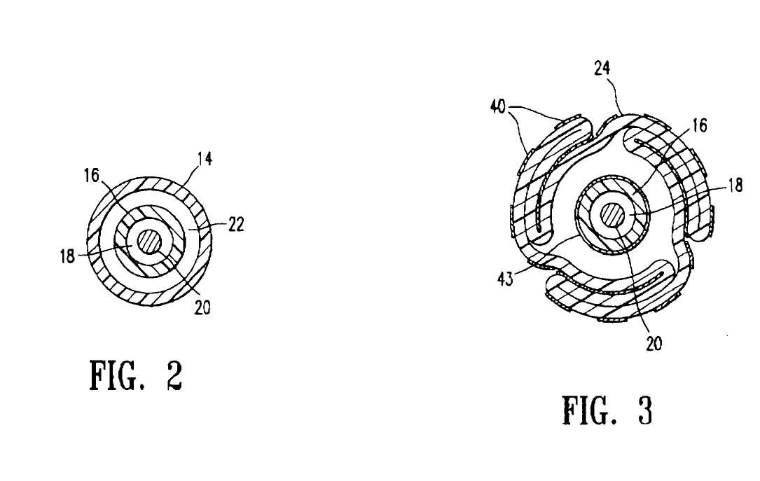

FIG. 1 illustrates an over-the-wire type stent delivery balloon catheter 10 embodying features of the invention. Catheter 10 generally comprises an elongated catheter shaft 12 having an outer tubular member 14 and an inner tubular member 16. Inner tubular member 16 defines a guidewire lumen 18 configured to slidingly receive a guidewire 20, and the coaxial relationship between outer tubular member 14 and inner tubular member 16 defines annular inflation lumen 22, as best shown in FIGS. 2 and 3 illustrating transverse cross sections of the catheter shown in FIG. 1, taken along lines 2-2 and 3-3, respectively. An inflatable balloon 24 disposed on a distal section of catheter shaft 12 has a proximal skirt section 25 sealingly secured to the distal end of outer tubular member 14 and a distal skirt section 26 sealingly secured to the distal end of inner tubular member 16, so that the balloon interior is in fluid communication with inflation lumen 22. An adapter 30 at the proximal end of catheter shaft 12 is configured to provide access to guidewire lumen 18, and to direct inflation fluid through arm 31 into inflation lumen 22. In the embodiment illustrated in FIG. 1, the balloon 24 is illustrated prior to complete inflation thereof, with an expandable stent 32 mounted on the working length of the balloon 24 for implanting within a patient's body lumen 27. The distal end of catheter 10 may be advanced to a desired region of the patient's body lumen 27 in a conventional manner, the balloon 24 inflated to expand stent 32, and the balloon deflated, leaving the stent 32 implanted in the body lumen 27.

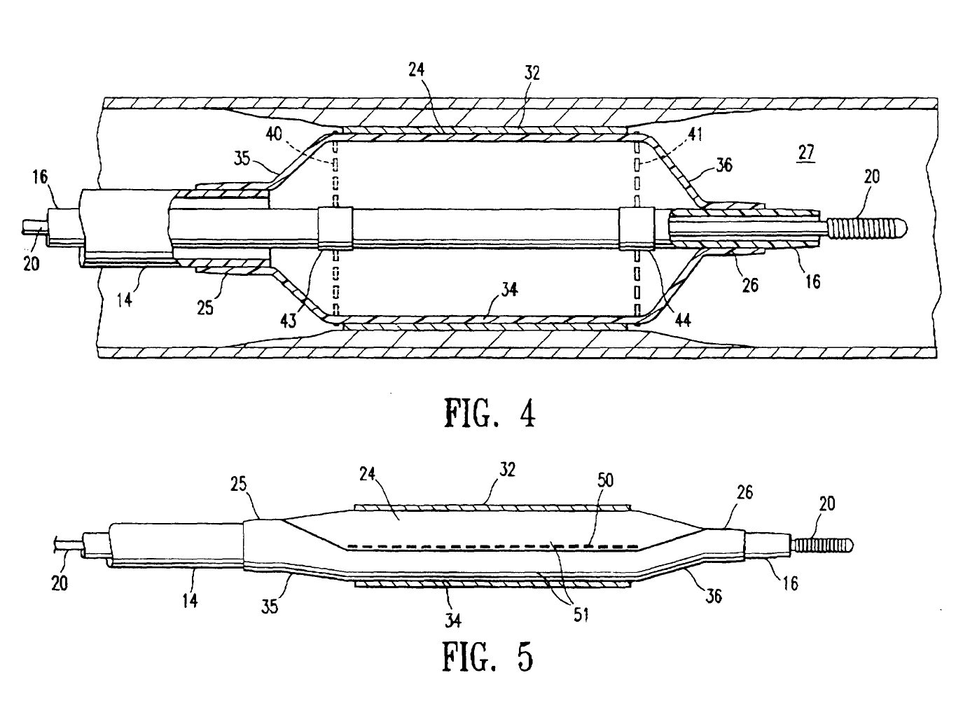

FIG. 4 illustrates the distal end of the balloon catheter of FIG. 1, with the balloon 24 fully inflated in the body lumen 27 to expand the stent 32. In the inflated configuration, balloon 24 has an inflated central working length 34, a proximal tapered section 35 between the inflated working length and the proximal skirt section 25, and a distal tapered section 36 between the inflated working length and the distal skirt section 26.

In the embodiment of FIG. 1, the balloon 24 has a proximal visible mark 40 on the outer surface of the balloon at the proximal end of the working length 34, and a distal visible mark 41 on the outer surface of the balloon at the distal end of the working length 34. In the embodiment of FIG. 1, the proximal and distal visible marks are an ink coating. A variety of suitable inks may be used to form the ink coating mark, which are typically indelible and biocompatible. However, a variety of suitable visible marks may be used including a titanium oxide mark and a scorch mark as discussed in more detail below.

The ink marks are preferably applied by a method selected from the group consisting of wiping, spraying, masked dipping (i.e., dipping in ink with part of the balloon surface masked to prevent being coated by the ink), and electrostatic attraction. For example, the ink can be wiped onto the outer surface of the balloon using a felt applicator, sponge or other applicator. Alternatively, the ink can be sprayed onto the outer surface of the balloon using a compressed air sprayer, or an ink jet applicator which atomizes the ink. The thickness of the ink marks 40, 41 are exaggerated somewhat in FIG. 3 for ease of illustration. In one embodiment, the ink marks 40, 41 have a dimension of about 0.1 mm to 0.3 mm.

A scorch mark typically involves exposing the outer surface of the balloon to laser radiation, using a conventional laser at a sufficient power level and duration to discolor the balloon material. The laser scorch marks are provided with a desired width and thickness based on the laser operating conditions used and duration of exposure.

A titanium oxide mark is typically formed by exposing titanium dioxide in or on the balloon to an ultraviolet laser. In a presently preferred embodiment, the titanium dioxide is blended with the balloon polymeric material to form a titanium dioxide loaded balloon. The titanium dioxide-polymeric material blend can be used to form a single layer balloon, or a first layer of a multilayered balloon which typically involves coextruding the titanium dioxide-polymeric material blend as an outer layer with an inner layer of a polymeric material free of titanium dioxide. The balloon is exposed to an ultraviolet laser to convert the exposed titanium dioxide to darkened titanium oxide in a desired pattern such as the circumferentially extending dashed bands 40, 41 illustrated in FIG. 1. Depending on the percent loading of the titanium dioxide, the resulting visible marks are light gray to black. In one embodiment, the percent loading of the titanium dioxide in the titanium dioxide-polymeric material blend is about 0.5% to about 2%, more specifically about 1% in order to provide a sufficiently visible mark without disadvantageously affecting the balloon as, for example, by producing pin-hole defects in the balloon. The ultraviolet laser does not produce any disadvantageous heating of the balloon polymeric material, and the resulting mark is indelible unless polymeric material of the balloon is removed.

In the embodiment of FIG. 1, the visible marks 40, 41 are discontinuous dashed lines extending completely around the circumference of the balloon in the deflated and inflated configurations illustrated in FIGS. 1 and 4. However, the visible marks 40, 41 can have a variety of suitable configurations including a continuous solid line, or a dotted line. In the embodiment of FIG. 1, the balloon 24 has no more than two visible marks (i.e., proximal and distal visible marks 40 and 41), so that the balloon is not provided with visible marks on a central section of the balloon located between the proximal and distal visible marks 40, 41. The visible marks 40, 41 are radially aligned with markers 43, 44 on the inner tubular member in the interior of the balloon 24. Markers 43, 44 are typically radiopaque markers for catheter visualization using fluoroscopy, although a variety of suitable catheter visualization system markers may be used including MRI visualization. Therefore, although discussed below primarily in terms of an embodiment in which markers 43, 44 are radiopaque markers, it should be understood that alternative visualization system markers may be used. In the embodiment of FIG. 1, the visible marks 40, 41 are radially aligned with the center of the radiopaque markers 43, 44. However, in alternative embodiments (not shown), the visible marks 40, 41 are aligned with the radiopaque markers 43, 44 in a variety of suitable configurations as are conventionally known for indicating the location of the balloon in the patient's body lumen. For example, the visible marks 40, 41 can alternatively be aligned with an end of the radiopaque markers 43, 44, or a single radiopaque marker can be aligned with the center of the working length 34 of the balloon 24.

In the embodiment in which the visible marks 40, 41 are ink marks, the ink marks 40, 41 are typically applied to the balloon 24 in the inflated configuration, although the ink marks 40, 41 may alternatively be applied to the balloon in the deflated configuration. In the embodiment in which the visible marks 40, 41 are laser scorch marks, the scorch marks are typically applied to the balloon 24 in the deflated configuration, which facilitates focusing the laser focal point at the desired location on the thin wall of the balloon 24. In the embodiment in which the marks 40, 41 are formed on the balloon 24 in the inflated configuration, the proximal skirt section 25 of the balloon 24 is typically secured to the outer tubular member 14 before the marks 40, 41 are made on the balloon 24, to facilitate inflating the balloon 24.

The location for the visible marks 40, 41 at either end of the working length 34 of the balloon 24 is determined by engineering and the design of the catheter. Thus, the balloon 24 is formed, as for example by blow molding, with known dimensions, allowing the required location of the balloon shoulders to be determined by measuring a specified distance from a given set point. For example, the working length 34 has a specified length, and the proximal end of the working length can be specified by its distance from the proximal end of the balloon. After the desired location for the proximal and distal visible marks 40, 41 is thus located, the marks 40, 41 are made on the outer surface of the balloon 24. In one embodiment, the marks 40, 41 facilitate positioning the balloon 24 at a desired location on the shaft 11 relative to the radiopaque markers 43, 44 on the inner tubular member 16, and thus the proximal and distal visible marks 40, 41 are made on the balloon 24 before the catheter 10 is fully assembled. During assembly of the catheter 10, the technician aligns the radiopaque markers 43, 44 with the visible marks 40, 41, and then secures the distal skirt section 26 of the balloon 24 to the thus aligned inner tubular member 16.

In one embodiment, the proximal and distal visible marks 40, 41 on the ends of the working length 34 of balloon 24 facilitate correctly positioning the stent 32 on the working length 34 of the balloon 24. The technician mounts the stent 32 on the balloon between the proximal and distal visible marks 40, 41, typically with the ends of the stent a given distance from the visible marks 40, 41. In the embodiment in which the proximal and distal visible marks 40, 41 are made on the balloon 24 to mark the desired stent position on the balloon 24, the marks 40, 41 can be made before or after the catheter 10 is fully assembled (e.g., before or after the balloon skirt sections 25, 26 are secured to the outer tubular member 14 and to the inner tubular member 16, respectively). A variety of commercially available stents may be used with the balloon catheter of the invention, typically comprising expandable metal tubes. See for example, U.S. Pat. No. 5,507,768 (Lau et al.) and U.S. Pat. No. 5,458,615 (Klemm et al), which are incorporated herein by reference. The stent 32 is mounted on the balloon 24 as conventionally known by crimping or otherwise releasably securing the stent for delivery and deployment within a patient's body lumen.

FIG. 5 illustrates an alternative embodiment of the invention, having visible mark 50 on the balloon 24 outer surface at the edge of a folded wing 51 wrapped around the balloon 24 in the deflated configuration. The folded balloon 24 in the deflated configuration preferably has three wings 51 wrapped around the balloon 24 to form a low profile configuration for introduction and advancement within the patient's body lumen. The edge the balloon wing 51 is marked with the visible mark 50 before the stent 32 is mounted on the balloon 24, and typically after the balloon proximal and distal skirt sections 25, 26 of the balloon 24 are secured to the shaft 11. The balloon 24 is pressed and twisted in the deflated configuration, so that the wing mark 50 facilitates determining the amount of twist introduced into the deflated folded balloon. Similarly, the wing mark 50 facilitates determining the amount of pinching of the wing 51. The discussion above relating to the visible marks 40, 41 applies as well to visible mark 50 on the balloon wings 51. Preferably, each wing 51 of the balloon has visible mark 50 extending longitudinally along the length of the balloon at the edge of the wing. The edge of the wing extends longitudinally along the length of the balloon on the exposed outer surface of the wing 51 at the point between the exposed outer surface of the wing 51 and the unexposed surface of the wing 51 formed by the fold of the wing 51.

Although the balloon is illustrated with a conventional inflated configuration having a central cylindrical working length between two gradually tapered inflatable sections, it should be understood that the inflated balloon may have a variety of suitable configurations including balloon configurations specially shaped for a particular anatomy such as a focal balloon configuration, a conical balloon configuration, and the like, as are conventionally known to one of skill in the art.

The dimensions of catheter 10 are determined largely by the size of the balloon and guidewire to be employed, the catheter type, and the size of the artery or other body lumen through which the catheter must pass or the size of the stent being delivered. Typically, the outer tubular member 14 has an outer diameter of about 0.025 to about 0.04 inch (0.064 to 0.10 cm), usually about 0.037 inch (0.094 cm), and the wall thickness of the outer tubular member 14 can vary from about 0.002 to about 0.008 inch (0.0051 to 0.02 cm), typically about 0.003 to 0.005 inch (0.0076 to 0.013 cm). The inner tubular member 16 typically has an inner diameter of about 0.01 to about 0.018 inch (0.025 to 0.046 cm), usually about 0.016 inch (0.04 cm), and a wall thickness of about 0.004 to about 0.008 inch (0.01 to 0.02 cm). The overall length of the catheter 10 may range from about 100 to about 150 cm, and is typically about 143 cm. Preferably, balloon 24 has a length about 0.8 cm to about 6 cm, and an inflated working diameter of about 2 to about 10 mm.

Inner tubular member 16 and outer tubular member 14 can be formed by conventional techniques, for example by extruding and necking materials already found useful in intravascular catheters such a polyethylene, polyvinyl chloride, polyesters, polyamides, polyimides, polyurethanes, and composite materials. The various components may be joined using conventional bonding methods such as by fusion bonding or use of adhesives. Although the shaft is illustrated as having coaxial inner and outer tubular members, a variety of suitable shaft configurations may be used including a dual lumen extruded shaft having side-by-side lumens along at least a section thereof. Similarly, although the embodiment illustrated in FIG. 1 is an over-the-wire stent delivery catheter, balloons of this invention may also be used with other types of intravascular catheters, such as rapid exchange type balloon catheters. Rapid exchange type catheters generally comprise an elongated shaft with a distal guidewire port in a distal end of the catheter, a proximal guidewire port in a distal shaft section located distal of the proximal end of the shaft and typically spaced a substantial distance from the proximal end of the catheter, and a short guidewire lumen extending between the proximal and distal guidewire ports in the distal shaft section.

While the present invention is described herein in terms of certain preferred embodiments, those skilled in the art will recognize that various modifications and improvements may be made to the invention without departing from the scope thereof. Moreover, although individual features of one embodiment of the invention may be discussed herein or shown in the drawings of the one embodiment and not in other embodiments, it should be apparent that individual features of one embodiment may be combined with one or more features of another embodiment or features from a plurality of embodiments.

What is claimed:

1. A balloon catheter, comprising
a) an elongated shaft having at least one lumen therein; and
b) a balloon on a distal shaft section having a proximal and a distal skirt section secured to the elongated shaft so that an inflatable interior of the balloon is in fluid communication with the shaft lumen, and having at least one visible mark visible from an outer surface of the balloon, the visible mark being selected from the group consisting of an ink coating mark, a titanium oxide mark, and a scorch mark.

2. The balloon catheter of claim 1 wherein the visible mark is nonradiopaque.

3. The balloon catheter of claim 1 wherein the visible mark is aligned with a visualization system marker on a portion of the shaft extending through the inflatable interior of the balloon.

4. The balloon catheter of claim 1 wherein the balloon has an inflated configuration with an inflated working length, a proximal tapered section between the inflated working length and the proximal skirt section of the balloon, and a distal tapered section between the inflated working length and the distal skirt section of the balloon, and the balloon has a proximal visible mark at a proximal end of the working length, and a distal visible mark at a distal end of the working length.

5. The balloon catheter of claim 4 wherein the catheter shaft comprises an inner tubular member extending through the inflatable interior of the balloon, the inner tubular member having two or more visualization system markers, and the proximal and distal visible marks are radially aligned with the visualization system markers of the inner tubular member.

6. The balloon catheter of claim 4 wherein the balloon catheter is a stent delivery catheter with a stent mounted on a working length of the balloon for delivery and deployment within a patient's body lumen, and the proximal visible mark is located at or proximally adjacent to a proximal end of the stent and the distal visible mark is located at or distally adjacent to a distal end of the stent.

7. The balloon catheter of claim 4 wherein the proximal and distal marks extend at least partially around the circumference of the balloon.

8. The balloon catheter of claim 4 wherein the proximal and distal marks extend completely around the circumference of the balloon.

9. The balloon catheter of claim 4 wherein the visible marks are not located at a central section of the balloon located between the proximal and distal visible marks.

10. The balloon catheter of claim 1 wherein the balloon has a deflated configuration with one or more wings wrapped around the balloon before inflation of the balloon in a patient's body lumen, and the visible mark extends along an edge of the wing.

11. The balloon catheter of claim 1 wherein the visible mark is a dashed, dotted, or continuous line.

12. A balloon catheter, comprising:
a) an elongated shaft having a proximal end, a distal end, and an inflation lumen; and
b) a balloon on a distal shaft section, having an interior in fluid communication with the inflation lumen of the shaft, and having a proximal and a distal skirt section bonded to the shaft, an inflated configuration and a deflated configuration, an inflated working length and a proximal inflated tapered section and a distal inflated tapered section in the inflated configuration, and a proximal visible mark at a proximal end of the working length and a distal visible mark at a distal end of the working length, the proximal and distal visible marks extending at least partially around the balloon circumference and being a nonradiopaque material.

13. The balloon catheter of claim 12 wherein the proximal and distal visible marks are selected from the group consisting of an ink coating mark, a titanium oxide mark, and a scorch mark.

14. A method of making a balloon catheter having an elongated shaft and a balloon on a distal shaft section, comprising making at least one visible mark visible from an outer surface of the balloon using a method selected from the group consisting of scorching the balloon material to form a scorch mark, coating the balloon material with an ink to form an ink coating mark, and converting titanium dioxide loaded in a wall of the balloon to titanium oxide to form a titanium oxide mark.

15. The method of claim 14 wherein the balloon has an inflated configuration with an inflated working length, a proximal inflated tapered section between the inflated working length and a proximal skirt section of the balloon, and distal inflated tapered section between the inflated working length and a distal skirt section of the balloon, and making the visible mark comprises forming a proximal visible mark at a proximal end of the working length and a distal visible mark at a distal end of the working length.

16. The method of claim 15 wherein the elongated shaft comprises an inner tubular member defining a guidewire lumen and an outer tubular member defining an inflation lumen, and including, after the proximal and distal visible marks are made, providing the shaft inner tubular member extending through an interior of the balloon and radially aligning visualization system markers on the inner tubular member with the proximal and distal visible marks on the balloon to align the inner tubular member, and securing the distal skirt section of the balloon to the aligned inner tubular member.

17. The method of claim 15 wherein the proximal and distal visible marks are made before or after the balloon proximal and distal skirt section are secured to the shaft, and including, after the marks are formed, mounting a stent on the balloon working length with the stent between the proximal and distal visible marks.

18. The method of claim 15 including inflating the balloon to the inflated configuration before the proximal and distal visible marks are made, and wherein the proximal and distal visible marks are formed on the balloon in the inflated configuration.

19. The method of claim 15 wherein the proximal and distal visible marks comprise scorch marks, and the scorch marks are formed on the balloon in a deflated configuration.

20. The method of claim 14 wherein the balloon has an inflated configuration with an inflated working length, a proximal inflated tapered section between the inflated working length and a proximal skirt section of the balloon, and distal inflated tapered section between the inflated working length and a distal skirt section of the balloon, and wherein the visible mark is formed along an edge of a wing of the balloon in a deflated folded configuration after the balloon proximal and distal skirts sections are secured to the elongated shaft.

21. The method of claim 20 including pressing and twisting the deflated folded balloon after the visible mark is formed.

22. The method of claim 20 including mounting a stent on the working length of the balloon after the visible mark is formed.

23. The method of claim 14 wherein the visible mark is nonradiopaque and the mark is formed as a dashed, dotted, or continuous line.

24. The method of claim 14 wherein the visible mark is ink applied to the balloon by a method selected from the group consisting of wiping, spraying, masked dipping, and electrostatic attraction.

25. The method of claim 14 wherein the visible mark is scorched balloon material formed by exposing the balloon to laser radiation.

26. The method of claim 14 wherein the titanium dioxide loaded in the wall of the balloon is converted to titanium oxide by exposing a portion of the balloon to ultraviolet radiation to form the titanium oxide mark.

27. A stent delivery balloon catheter, comprising:
a) an elongated shaft having a proximal end, a distal end, and an inflation lumen; and
b) a balloon on a distal shaft section, having an interior in fluid communication with the inflation lumen of the shaft, and having a proximal and a distal skirt section bonded to the shaft, an inflated configuration and a deflated configuration, a working length with an expandable stent mounted on the working length, a proximal visible mark proximal to a proximal end of the stent, and a distal visible mark distal to a distal end of the stent, the proximal and distal visible marks extending at least partially around the balloon circumference and being a nonradiopaque material.

28. The stent delivery balloon catheter of claim 27 wherein the visible marks are selected from the group consisting of an ink coating mark, a titanium oxide mark, and a scorch mark.
